(19) United States
(12) Patent Application Publication
(10) Pub. No.: US 2023/0237242 A1
(43) Pub. Date: Jul. 27, 2023

(54) SYSTEMS AND METHODS FOR GENERATING EMOTIONALLY-ENHANCED TRANSCRIPTION AND DATA VISUALIZATION OF TEXT

(71) Applicants: JOSEPH SEROUSSI, TEL-AVIV (IL); DORON TABOH, REHOVOT (IL)

(72) Inventors: JOSEPH SEROUSSI, TEL-AVIV (IL); DORON TABOH, REHOVOT (IL)

(21) Appl. No.: 18/011,537

(22) PCT Filed: Jun. 24, 2021
(86) PCT No.: PCT/IB2021/055597

§ 371 Date: Dec. 20, 2022

Related U.S. Application Data
(60) Provisional application No. 63/043,207, filed on Jun. 24, 2020.

(51) Int. Cl. G06F 40/103; G06F 40/40; G06F 40/232; G06V 40/20; G06V 40/16

(52) U.S. Cl. CPC G06F 40/103; G06F 40/40; G06F 40/232; G06V 40/20; G06V 40/176

(57) ABSTRACT

Generating emotionally enhanced transcription of non-textual data and an enriched visualization of transcribed data by capturing non-textual data of a speaker using bio-feedback technology, transcribing it into to a textual format, combining transcribed textual data with emotional state of the speaker to generate the emotionally enhanced transcribed textual data, and presenting emotionally enhanced transcribed textual data through an enriched visualization including color-coding transcribed textual data to identify mistakes in the transcribed data.

### FIELD AND BACKGROUND OF THE DISCLOSURE

The disclosure herein relates to systems and methods for generating emotionally-enhanced transcriptions and data visualization of text from audio and visual data.

Therapy has traditionally maintained a technology-free approach in which a therapist meets a patient in a room for a set amount of time. While there are many pros to maintaining such an approach, therapy can become much more efficient through the introduction of technological tools.

The use of video chat for purposes other than personal chats is growing through the increased video-chat capabilities and the experience of COVID-19 which forcibly introduced many people to video-chats. Video chat is booming and with it are transcription tools which transcribe voice/video to text. Transcription of video chats will become more important as the use of video chats increases.

However, transcription from video chat is still not 100% and requires fixing or completing texts manually which can be time-consuming. Although transcription technology is improving rapidly, it still remains flawed achieving around 85% successful transcription. This requires going back to the original video chat and searching for the appropriate footage in order to fix or complete the transcription.

Another weakness in transcribed text in some cases is that it requires a lot of time to read through the text of a whole session. In the case of therapists, reading the transcript of a past session may be too time-consuming for the therapist to be able to focus upon the more meaningful parts of the transcript.

Video chat allows us to communicate using voice, but it also includes body language which may offer us hints as to the emotional state of the person we are communicating with. All humans are adept at understanding some body language, but this is a skill which few really master. Even the best therapist can miss subtle hints as to the emotional state of a patient. Furthermore, it is nearly impossible through traditional therapy to search, track and analyze the efficiency of the therapy—identifying patterns over time, understanding whether the therapy is succeeding and by how much.

The transcription of a video-audio chat does not offer easily accessible hints as to the “subtext” (the “in-between-the-lines”) the emotional state of the person who spoke in the chat. The Bio-feedback technologies such as Voice Analysis or Voice Stress Analysis (VSA) which test the amount of stress in the voice of the speaker and Facial Macro-Micro Expressions (FMME) technologies which identify up to 21 different emotions exist band can help to tap into the subtext which underlies what is being spoken. However, these Bio-feedback technologies have not been integrated with the audio/video chats nor transcription to provide better information about the patient to the therapist.

Since time is a key factor in audio and video chats, the therapist may not have the time to read the complete text and might miss out some important information. The visualization of the transcribed text has primarily remained ordinary providing no information on the key focus points.

In light of the above shortcomings, there is a need to improve the visualization of audio and video transcription while integrating the emotional aspects of the patient.

### SUMMARY OF THE EMBODIMENTS

In one aspect of the invention, a method for generating emotionally enhanced transcription of non-textual data and an enriched visualization of the transcribed data is disclosed. The method comprises the steps of capturing the non-textual data of a speaker involved in a conversation and transcribing the non-textual data to generate a textual data. The method further comprises obtaining an emotional state of the speaker using one or more bio-feedback technologies and combining the generated transcribed textual data with the emotional state of the speaker to generate BIOsubTEXT, the emotionally enhanced transcribed textual data. The method also comprises presenting the emotionally enhanced transcribed textual data through an enriched visualization, wherein the enriched visualization includes color-coding, tempo-coding and weight-coding the emotionally enhanced transcribed textual data.

In an another aspect of the invention, the non-textual data comprises a video or an audio conversation or a combination thereof between the speaker and a user.

In an another aspect of the invention, the bio-feedback technologies include one or more of a Voice Sensitivity Analysis, Voice Stress Analysis, Facial Macro-Micro Expressions (FMME) technologies, Layered Voice Analysis, Infra-Red (heat) analysis and Oximeter (pulse) analysis.

In an another aspect of the invention, color-coding the emotionally enhanced transcribed textual data comprises color-coding the transcribed textual data based on its level of certainty, confidence and stress of the speaker(s).

In an another aspect of the invention, color-coding the emotionally enhanced transcripted textual data comprises color-coding the transcripted textual data based on a level of stress of the speaker.

In an another aspect of the invention, the method further comprises linking the emotionally enhanced transcribed textual data to a video-audio timeline, wherein the video-audio timeline enables easy access of the non-textual data and its emotionally enhanced transcribed textual data. Connecting video, audio, text and emotions on one time-line offers the therapist, or the manager, an efficient tool to manage all the different aspects of communication together—one can access the video from the text, the emotions from the text, the text from the audio etc.

In an another aspect of the invention, the enriched visualization includes presenting the emotionally enhanced transcribed textual data through different color, size, weighting and spacing of the text.

In an another aspect of the invention, the enriched visualization further comprises zooming out of the transcribed textual data to identify hot-spot areas and zooming in to the text in the hot-spot areas. This is a crucial part of the invention since it offers therapists and managers a tool to quickly identify the parts of the text which may be more significant.

In an another aspect of the invention, the method further comprises using alternative therapy tools to fine tune the quality of the emotionally enhanced transcribed textual data.

In an another aspect of the invention, the method further comprises using artificial intelligence to search, track and analyse the patterns and correlation between the transcribed textual data and the emotions of the speaker in an audio or a video conversation.

In an another aspect of the invention, the method further comprises using machine learning to search, track and analyse the correlation between the transcribed textual data and the emotions of the speaker by comparing the audio or the video conversation with previously stored conversations.

In an another aspect of the invention, the method further comprises using one or more emojis, emotionally enhanced graphics, along with the transcribed textual data to identify the emotional state of the speaker.

In an another aspect of the invention, the method further comprises a fL0Ow text mechanism, wherein the fL0Ow text mechanism is a Tempo-Spaced Text Mechanism configured to use the tempo of the sound-track and Micro-Expression analysis of the speaker in an audio or a video conversation to identify the emotional state of the speaker.

In an another aspect of the invention, the fL0Ow text mechanism includes presenting the emotionally enhanced transcribed textual data through different levels of font, letter and word spacing, boldness, italicizing, weighting of the text to identify the tempo of the speaker.

In an another aspect of the invention, the enriched visualization further comprises zooming out of the transcribed textual data to identify areas of different levels of tempo and zooming in to identify specific textual data related to the tempo.

In an another aspect of the invention, the method further comprises providing a Customer Relations Management (CRM) tool, wherein the CRM tool enables multi-channel communication between the speaker and a user involved in an audio or a video conversation.

In an another aspect of the invention, a system for generating emotionally enhanced transcription of non-textual data and an enriched visualization of the transcribed data is disclosed. The system comprises a receiving module configured for receiving the non-textual data of a speaker involved in a conversation, a transcription module configured for transcribing the non-textual data to generate a textual data and a bio-feedback module configured for obtaining an emotional state of the speaker using one or more bio-feedback technologies. The system further comprises an analysis module configured for combining the generated transcribed textual data with the emotional state of the speaker to generate the emotionally enhanced transcribed textual data and a visual presentation module configured for presenting the emotionally enhanced transcribed textual data through an enriched visualization, wherein the enriched visualization includes color-coding, tempo-coding and weight-coding the emotionally enhanced transcribed textual data.

In an another aspect of the invention, the visual presentation module is further configured for presenting the emotionally enhanced transcribed textual data through different color, size, weighting and spacing of the text.

In an another aspect of the invention, the system further comprises alternative therapy tools to fine tune the quality of the emotionally enhanced transcribed textual data.

In an another aspect of the invention, the system further comprises an artificial intelligence module for searching, tracking and analysing the correlation between the transcribed textual data and the emotions of the speaker in an audio or a video conversation, a memory module for storing the transcribed textual data and the emotions of the speaker and a machine learning module for comparing the audio or the video conversation with previously stored conversations for fine tuning the analysis module.

In an another aspect of the invention, the fL0Ow text mechanism module is further configured for presenting the emotionally enhanced transcribed textual data through different levels of font, letter and word spacing, boldness, italicizing, weighting of the text to identify the tempo of the speaker.

In an another aspect of the invention, the fL0Ow text mechanism module is further configured for zooming out of the transcribed textual data to identify areas of different levels of tempo and zooming in to identify specific textual data related to the tempo.

### BRIEF DESCRIPTION OF THE FIGURES

For a better understanding of the embodiments and to show how it may be carried into effect, reference will now be made, purely by way of example, to the accompanying drawings.

With specific reference now to the drawings in detail, it is stressed that the particulars shown are by way of example and for purposes of illustrative discussion of selected embodiments only, and are presented in the cause of providing what is believed to be the most useful and readily understood description of the principles and conceptual aspects. In this regard, no attempt is made to show structural details in more detail than is necessary for a fundamental understanding; the description taken with the drawings making apparent to those skilled in the art how the various selected embodiments may be put into practice. In the accompanying drawings:

FIG. 1 illustrates an exemplary conversation environment between a patient and a therapist according to an aspect of the invention;

FIG. 2A is an illustrative representation of elements of an emotionally enhanced transcription system;

FIGS. 2B and 2C illustrates an example of a sample from a transcribed text and a corresponding an emotionally-enhanced BIOsubTEXT.

FIG. 3 is a block diagram illustrating the structure of aspects of the emotionally enhanced transcription system;

FIGS. 4A and 4B a flowchart illustrating the method steps according to an aspect of the invention;

FIG. 5 is a block diagram illustrating the aspects of enriched visualization;

FIG. 6 illustrates an exemplary system for implementing various aspects of the invention; and

FIGS. 7A-F illustrate possible graphic user interfaces for an emotion enhanced therapy visualization system.

### DETAILED DESCRIPTION

Aspects of the present disclosure relate to generating enhanced transcriptions of non-textual data, particularly audio and visual data. In particular, the disclosure relates to generating emotionally enhanced transcription of non-textual data and an enriched visualization of the transcribed data.

In various embodiments of the disclosure, one or more tasks as described herein may be performed by a data processor, such as a computing platform or distributed computing system for executing a plurality of instructions. Optionally, the data processor includes or accesses a volatile memory for storing instructions, data or the like. Additionally or alternatively, the data processor may access a non-volatile storage, for example, a magnetic hard-disk, flash-drive, removable media or the like, for storing instructions and/or data.

It is particularly noted that the systems and methods of the disclosure herein may not be limited in its application to the details of construction and the arrangement of the components or methods set forth in the description or illustrated in the drawings and examples. The systems and methods of the disclosure may be capable of other embodiments, or of being practiced and carried out in various ways and technologies.

Alternative methods and materials similar or equivalent to those described herein may be used in the practice or testing of embodiments of the disclosure. Nevertheless, particular methods and materials are described herein for illustrative purposes only. The materials, methods, and examples are not intended to be necessarily limiting.

### Description of the Embodiments

According to various aspects of the current disclosure video-chat platforms are introduced which are enriched with transcription-based technologies and bio-feedback technologies in order to create emotionally-enhanced color-coded, weight-coded and/or tempo-coded BIOsubTEXT which empowers users to use the content more efficiently.

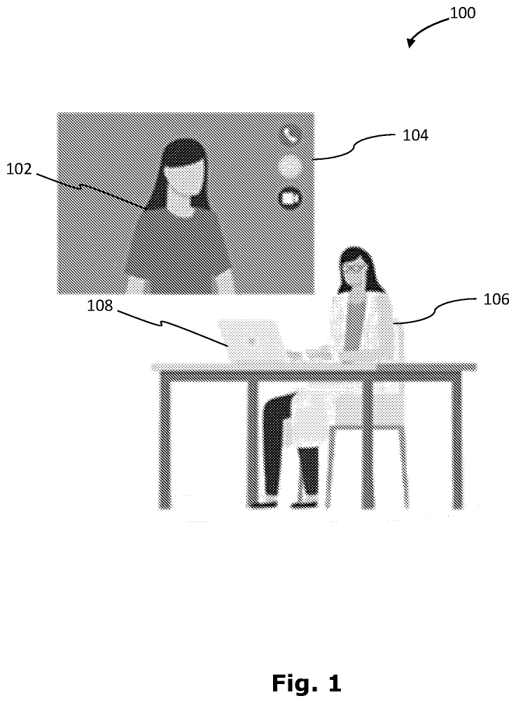

Reference is now made to FIG. 1 which illustrates an exemplary conversation environment 100 between a patient 102 and a therapist 106. The patient 102 and the therapist 106 are involved in a video conversation using their communication devices 104 and 108, respectively. The communication devices 104 and 108 may include, but not limited to, a personal computer, a server, a router, a network PC, a workstation, a microprocessor based appliance, a mobile phone, a laptop, a tablet, a paging device, a peer device or other common network node and the like. The communication devices 104 and 108 may communicate through a communication network (not shown) which may include a Bluetooth network, a Wired LAN, a Wireless LAN, a WiFi Network, a Zigbee Network, a Z-Wave Network, an Ethernet Network or a cloud network.

The patient 102 and the therapist 106 may be involved in a video chat wherein the therapist 106 may see the facial expressions and body language of the patient 102. Alternatively, the conversation between the patient 102 and the therapist 106 may be an audio chat wherein the therapist may not see the patient 102 and can only listen to her voice. During the chat, the patient 102 speaks about her physical and/or mental condition. The therapist 106 listen to the patient 102 and may note down some key points using an application on the communication device 108 or using a paper and a pen.

In still other embodiments, a face-to-face therapy session may be recorded by a audio and/or video recorder such that the data may be processed using the system and methods described herein.

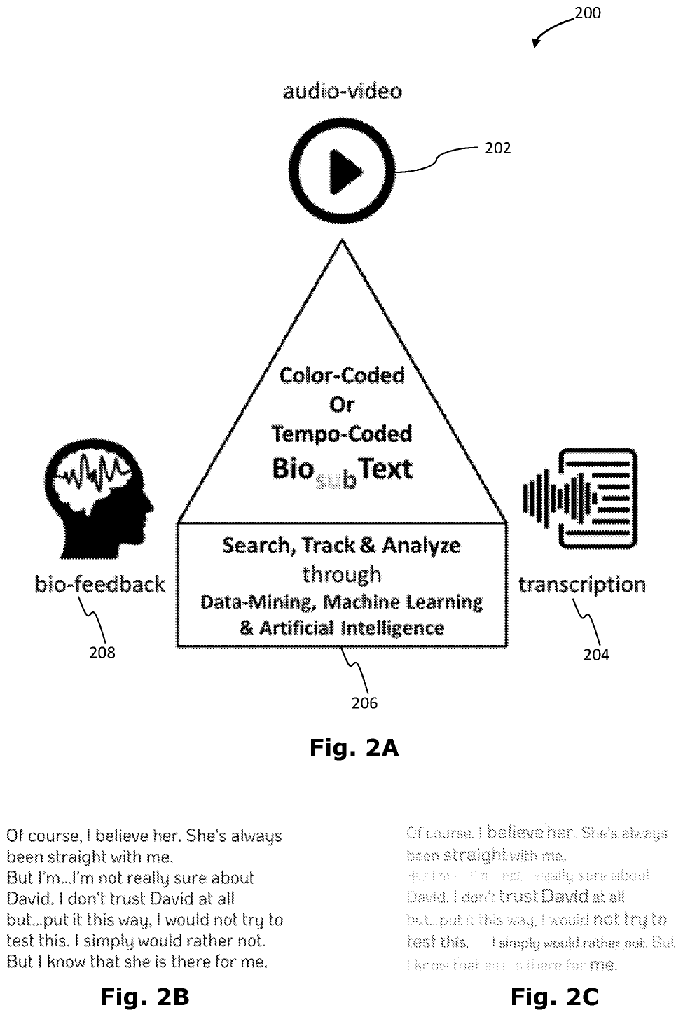

Reference is now made to FIG. 2 which is an illustrative representation 200 of the elements of an enhanced transcription system including:

Audio-Video Chat 202: The use of video chat for purposes other than personal chats is growing through the increased video-chat capabilities and the experience of COVID-19 which forcibly introduced many people to video-chats.

Transcription 204: Transcription from video chat is still not 100% and requires fixing or completing texts manually which can be time-consuming.

Text Search 206: Transcribed audio-video chats are much easier to search, track and analyze than the video chat itself.

Bio-Feedback 208: Video chats empowered with bio-feedback offer us hints as to the sub-text and the emotional state of the speaker through verbal, non-verbal and involuntary body language.

Versatility: These platforms will benefit therapists & patients, interviewers & interviewees, security officials (investigators, police, military . . . ) & the public, medical representatives & doctors, sales/service representatives & clients, people on dates etc. . . . These are all people whose lives rely heavily on social interactions based on one-on-one communications in a frontal, audio or video setting and the underlying importance of the emotions and the subtexts within the conversations. On the whole, communication in therapy remains as it has been since its inception: a frontal conversation between to people with little or no text apart from therapists' notes an no digital-data text.

For illustrative purposes only, FIG. 2B illustrates a sample from a transcribed text which may include all the verbal information spoken by a subject but does not provide any of the subtextual information carried by actual speech. For comparison FIG. 2C illustrates a corresponding emotionally-enhanced BIOsubTEXT of the same sample which uses color, size, weighting and spacing of the text to encode the subtextual information. It is a particular feature of the current system that the BIOsubTEXT presentation may allow therapists to readily focus on pertinent aspects of the transcript, such as particularly emotive terms, for example.

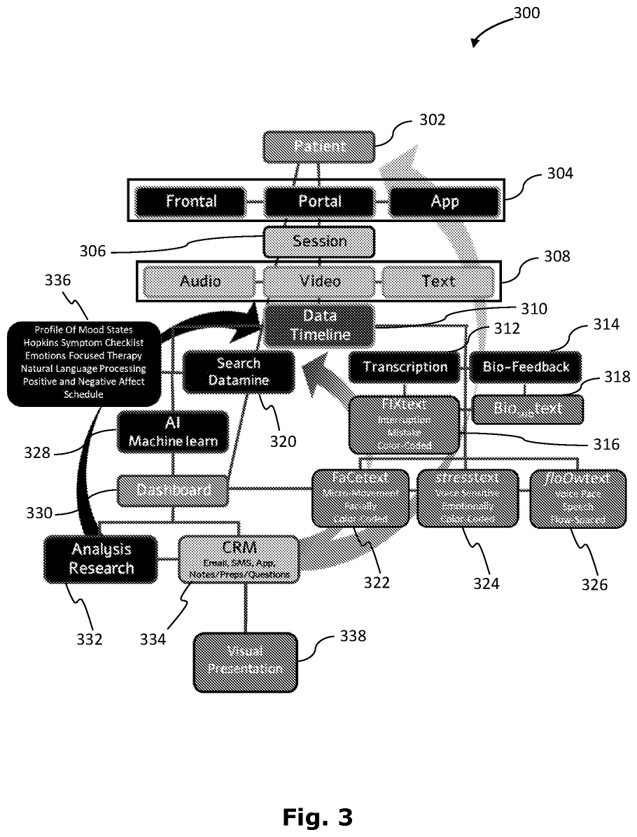

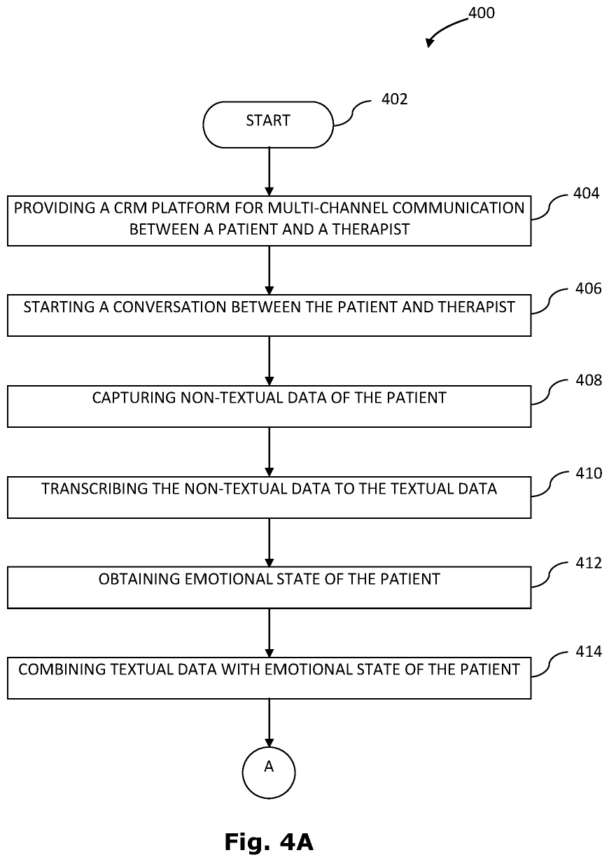

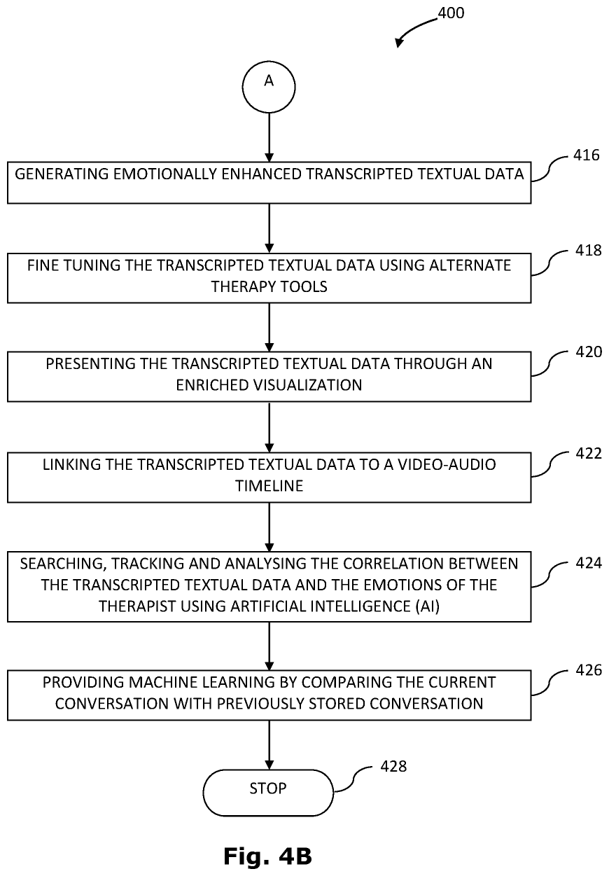

Referring now to FIG. 3 which is a structural block diagram illustrating aspects of the emotionally enhanced transcription system 300.

The system includes a patient 302 involved in a video conversation with a therapist 106. The patient 302 may connect with the therapist 106 using its communication device 104. The patient 302 may use various ways to connect online with the therapist 106. The patient 302 may use an online application or a portal 304, for example, a doctor consultation application or an application provided by a hospital, to connect with the therapist 106. Alternatively, the patient 302 and the therapist 106 may be involved in a frontal video conversation 304 via a video application, like Whatsapp, Skype, Google Meet, Microsoft Teams, Zoom Meeting, Cisco Webex Meet, etc.

The patient 302 and the therapist may be involved in a live video session 306. Alternatively, the session 306 may be a recorded video or audio clip sent by the patient 302 to the therapist 106. During the video session 306, the patient 302 speaks about her physical and/or mental condition. The patient provides the information 308 to the therapist 106 in form of an audio, a video and a text. The audio information may be provided through a microphone of the communication device 104 when the patient 302 speaks about her physical and/or mental condition. A video display of the communication device 104 enables the therapist 106 to receive facial expressions and body language of the patient 302. Further, the patient 302 may also provide text information in form of written message or send health reports to the therapist 106 via an email, SMS, etc.

The information provided by the patient 302 is received by a Data Timeline 310. The Data Timeline 310 is an audio/video/text information storage which records and stores the original information 304 received from the patient 302 in a timeline manner. For example, the information 304 may be stored as per the date of the information including the day, month and year. The time of the day may also be recorded for the information 304. Further, the information 304 may be stored in an ascending or descending order depending upon the date and time. The Data Timeline 310 enables easily access of the appropriate video-audio content by simply clicking on the text which may be provided in form of a patient's name, a patient serial number or any other form.

The patient information 308 is provided to a transcription module 312 which transcribes the non-textual audio and video information 308 into a textual format FIXtext 316. The transcription module 312 provides a FIXtext mechanism for Mistake-Based Color-Coded Text. In a particular embodiment, this mechanism uses color-coding to identify transcribed FIXtext 316 based on the uncertainty (or certainty) level of the transcribed text. For example, the FIXtext 316 which is uncertain is marked as “Orange” and non-transciptable FIXtext 316 is marked as “Red”. FIXtext mechanism also enables to zoom-in on to the text which needs to be fixed or completed to 100% transcription (eg: “I just can't understand {startFontOrange} weather {endFontOrange} I should fly out to {startFontRed} XXXX awe XX {endFontRed} or go back home”).

In an alternative embodiment, the intensity level of the marked color may provide an indication of the uncertainty level of the FIXtext 316. A sharp intensity color may indicate low level of certainty in the text and need to be fixed. Other colors may be preferred as appropriate.

The Data Timeline 310 may also store the transcribed FIXtext 316 along with the original information 308 provided by the patient 302. This will enable the therapist 106 to easily access the appropriate video-audio and its transcribed text.

Video chat allows us to communicate using voice but it also includes body language which may offer us hints as to the emotional state of the person we are communicating with. All humans are adept at understanding some body language but this is a skill which few really master. Bio-feedback technologies such as Voice Analysis or Voice Stress Analysis (VSA) which test the amount of stress in the voice of the speaker and Facial Macro-Micro Expressions (FMME) technologies which identify up to 21 different emotions exist band can help to tap into the subtext which underlies what is being spoken.

The emotionally enhanced transcription system 300 includes a Bio-Feedback module 314 which comprises a BIOsubTEXT mechanism for generating Emotionally Color/Space-Coded Text. The BIOsubTEXT mechanism uses bio-feedback technology during video chats in order to obtain hints and indications to the emotional state of the patient 302. The Bio-Feedback module 314 receives the facial expressions and body language information 308 of the patient 302 through the video display of the communication device 104 to generate a BIOsubTEXT 318 which represents the emotional state of the patient 302 using bio-feedback technologies. The bio-feedback technologies include, but not limited to, a Voice Sensitivity Analysis, Voice Stress Analysis, Facial Macro-Micro Expressions (FMME) technologies, Layered Voice Analysis, Infra-Red (heat) analysis and Oximeter (pulse) analysis.

The BIOsubTEXT information 318 received from the facial expressions and body language information 308 of the patient 302 is merged with the transcribed FIXtext 316 of the video-chat in the analysis module 332 in order to generate emotionally enhanced transcribed text. The emotionally enhanced transcribed text is presented through an enriched visualization which includes the emotional context of the spoken words through color, size, weighting and spacing of the text.

The emotionally enhanced transcribed text may be visually presented to the therapist 106 through a visual presentation module 338 of a Customer Relations Management (CRM) platform 334. The visual presentation module 338 may be a presentation module of the CRM 334 configured to display the transcribed text on the communication device 108 of the therapist 106. In a particular embodiment, the emotionally enhanced transcribed text uses green-yellow-orange-red color coding based on several bio-feedback parameters in order to offer therapists 106 and other users of the Customer Relations Management (CRM) platform 334 for an easy-to-understand view of the patient's 302 emotional state.

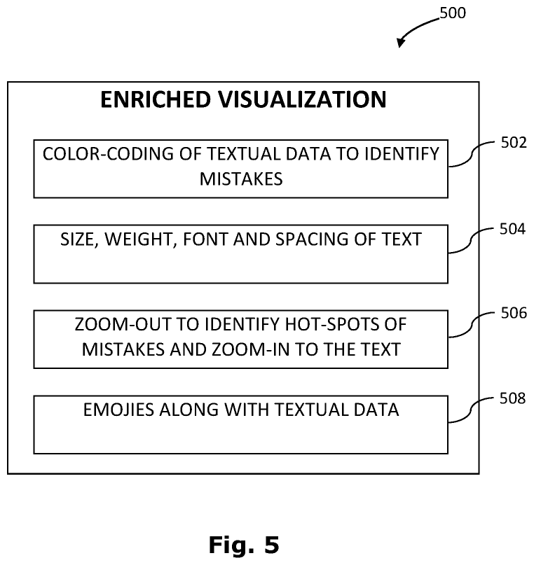

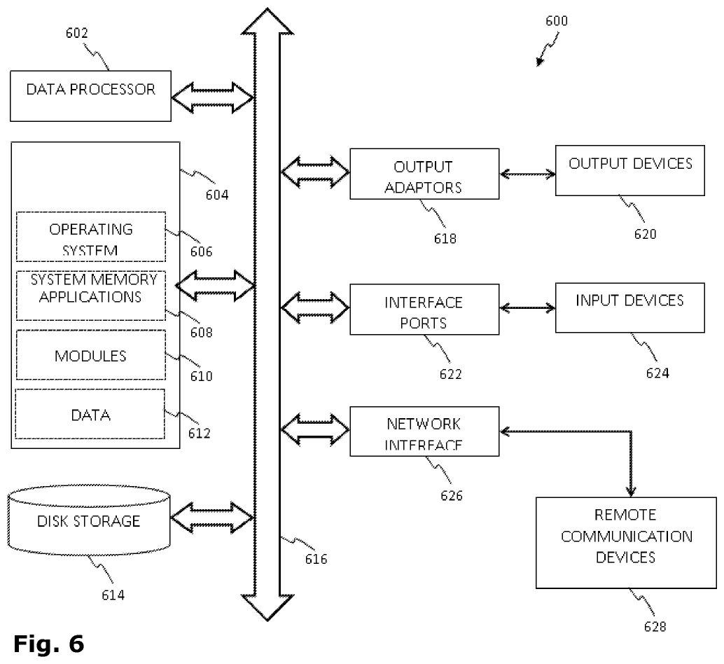

Referring to FIG. 5 which illustrates the various aspects of the enriched visualization 500. The enriched visualization 500 may include color-coding of textual data to identify mistakes 502 in the emotionally enhanced transcribed text. In a particular embodiment, the color-coding of textual data to identify mistakes 502 in the transcribed text may be based on the uncertainty (or certainty) level of the transcribed text. For example, the transcribed text which is uncertain is marked as “Orange” and non-transcribable text is marked as “Red”. In an alternative embodiment, the intensity level of the marked color may provide an indication of the uncertainty level of the transcribed text. A sharp intensity color may indicate low level of certainty in the text and need to be fixed. Other colors may be preferred as appropriate.

The enriched visualization 500 may also include the provision for modifying the size, weight, font and spacing of text 504 to identify mistakes in the transcribed text.

The enriched visualization 500 may also include the provision for zooming-out to identify the hot-spots of mistakes and zooming-in to the text 506 which needs to be fixed or completed to 100% transcription. For example, the statement may be presented as “I just can't understand {startFontOrange} weather {endFontOrange} I should fly out to {startFontRed} XXXX awe XX {endFontRed} or go back home”.

The enriched visualization 500 may also include the provision for presenting emojis along with the textual data 508 for easy recognition of the emotion of the patient 302.

The Data Timeline 310 may also store the emotionally enhanced transcribed text along with the transcribed FIXtext 316 and the original information 308 provided by the patient 302. This will enable the therapist 106 to easily access the appropriate video-audio and its transcribed text. Using tools to “zoom-out” of transcribed text to identify “hot-spots”, the areas in which the text has been color-coded red, and then “zoom-in” to the text itself. This is a key factor in data-visualization since the alternative is to view the whole video or read the whole text without clues as to the emotional state of the speakers acquired through bio-feedback.

Referring back to FIG. 3, the emotionally enhanced transcription system 300 may further include a FACEtext module 322 providing a FACEtext—Facially Color-Coded Text mechanism, STRESStext module 324 providing a STRESStext—Emotionally Color-Coded Text Mechanism and a fL0Owtext module 326 providing a fL0Ow text — Tempo-Spaced Text Mechanism. These mechanisms capture the emotional state of the patient 302 and helps to fine tune the quality of the emotionally enhanced transcribed text. The information captured from the FACEtext module 322, the stresstext module 324 and the fL0Owtext module 326 are provided to the analysis module 332 which fine tune the quality of the emotionally enhanced transcribed text.

The FACEtext module 322 provides a FACEtext—Facially Color-Coded Text mechanism. Such a mechanism may use facial Macro and Micro-Expression analysis to obtain hints as to specific emotions of the patient 302.

The FACEtext mechanism may further use color-coded highlighted transcribed text to easily identify words which hint at specific emotions. For example, a happy emotion may be highlighted in yellow, a sad emotion may be highlighted in blue, an angry emotion may be highlighted in red, a surprised emotion may be highlighted in pink, a disgusted emotion may be highlighted in purple etc.

In an alternative embodiment, the FACEtext mechanism may further use a series of emojis above or below the text for easy recognition of the emotion.

The FACEtext mechanism may further use zooming-out of the text to identify areas of different levels of stress by the color of the highlight and zooming-in to identify specific words related to the emotion.

The STRESStext module 324 provides a STRESStext—Emotionally Color-Coded Text Mechanism. The STRESStext mechanism uses Voice Analysis to obtain indicators as to the level of stress of the patient 302.

The STRESStext mechanism may further use color-coded transcribed text to easily identify words which hint at low levels of stress (green), medium levels of stress (orange) and high levels of stress (red).

The STRESStext mechanism may further use zooming-out of the text to identify areas of different levels of stress by the color of the text and zooming-in to identify specific words related to the level of stress.

The fL0Owtext module 326 provides a fL0Ow text—Tempo-Spaced Text Mechanism. The mechanism may use the tempo of the sound-track to the conversation and Micro-Expression analysis to obtain hints as to emotional context of the patient 302.

The fL0Ow text mechanism may further use different levels of font, letter and word spacing, boldness, italicizing and weighting to identify the tempo in which the text was spoken originally. For example, the fL0Ow text mechanism may represent a spoken word in flow of the voice tempo as “I'm not so sure anymore I'll have to think about this”.

The fL0Ow text mechanism may further use zooming-out of the text to identify areas of different levels of tempo and zooming-in to identify specific words related to the tempo.

The emotionally enhanced transcription system 300 may further use alternative therapy tools 336 to fine tune the quality of the emotionally enhanced transcribed textual data. The alternative therapy tools 336 may include, but not limited to, Natural Language Processing (NLP), Profile of Mood States (POMS), Hopkins Symptom Checklist (HSCL), Emotions Focused Therapy (EFT), Positive And Negative Affect Schedule (PANAS) and other scientifically-proved therapy tools to fine tune the quality of the emotionally enhanced transcribed text.

The emotionally enhanced transcription system 300 may further use Artificial Intelligence (AI) and Machine Learning (ML) 328 to search, track and analyze the correlation between the text (words) and the subtext (emotions) of the patient 302 in an audio or a video conversation. The Artificial Intelligence (AI) and Machine Learning (ML) 328 also enables “on the fly” learning of the transcription system 300 by comparing the current the audio or the video conversation with previously stored conversations by the same person or different people. The system 300 may employ any known machine learning algorithm, such as Deep Neural Networks, Convolutional Neural Networks, Deep Reinforcement Learning, Generative Adversarial Networks (GANs), etc. without limiting the scope of the invention.

The CRM platform 334 may be based on multi-channel communications such as e-mail, SMS, Apps etc. and allows the therapists 106 and patients 302 to communicate in between sessions. Through these channels, the therapist 106 can send the patient 302 questionnaires, training assignments, preparations before the next session, reports on past sessions etc. and the patient's behavior as a result of these will also become part of the data of the patient 302.

The therapist 106 may use the CRM platform 334 during a therapy session, to prepare for an upcoming session or for the purpose of research by using the following methodologies. For example:

Identifying emotional feelings in context to specific words during the session or over many sessions, or in comparison to other patients.

Identify the progress of the therapy sessions by tracking and analyzing the patient's language and emotions over time.

Track the effect of prescribed drugs or specific exercises over the emotional state of the patient over time.

Using the CRM platform 334, therapists 106 and the patients 302 can create and access reports of all kinds based on the data in order to accurately judge the progress of the therapy over time.

Therapists may use this platform to train and practice by identifying the therapists' ability to pick up on emotional cues of the patients during sessions.

Patients may use this platform for tasks issued by the therapist by logging and speaking into the platform. This may even become an integral part of self-therapy.

The acquired data of all the therapy sessions by all users can also be used for the purpose of research and market analysis. For example, the pharmaceutical companies can track and analyze the effects of drugs taken by patients, the therapists can compare the effects of different types of therapy on many patients, etc.

The data may also help to create profiles of therapists and patients and serve the platform managers to help match patients and therapists based on the successes and failures of therapists and patients of similar profiles.

Reference is now made to FIGS. 4A and 4B which illustrates a flowchart showing the method steps according to an aspect of the invention. The process starts at step 402 and a CRM platform is provided at step 404 for multi-channel communication between the patient 302 and the therapist 106. The multi-channel communication may be an audio or a video chat or text messaging. At step 408, the non-textual data of the patient 302 in form of audio and video is captured by the emotionally enhanced transcription system 300. At step 410, the captured audio and video data is transcribed to a textual form, FIXtext 316, by the transcription module 312. The emotional state of the patient 302 is captured by the Bio-Feedback module at step 412 to generate BIOsubTEXT 318. The generated FIXtext 316 and BIOsubTEXT 318 are combined at the analysis module 332 at step 414 to generate emotionally enhanced transcribed text at step 416.

The transcribed text is fine tuned using alternate therapy tools 336 at step 418. The alternative therapy tools 336 may include, but not limited to, Natural Language Processing (NLP), Profile of Mood States (POMS), Hopkins Symptom Checklist (HSCL), Emotions Focused Therapy (EFT), Positive And Negative Affect Schedule (PANAS) and other scientifically-proved therapy tools to fine tune the quality of the emotionally enhanced transcribed text.

The transcribed text is presented through an enriched visualization 500 of the CRM 334 at step 420. The enriched visualization 500 may include color-coding of textual data to identify mistakes 502 in the emotionally enhanced transcribed text. In a particular embodiment, the color-coding of textual data to identify mistakes 502 in the transcribed text may be based on the uncertainty (or certainty) level of the transcribed text. The enriched visualization 500 may also include the provision for modifying the size, weight, font and spacing of text 504. The enriched visualization 500 may also include the provision for zooming-out to identify the hot-spots of mistakes and zooming-in to the text 506 which needs to be fixed or completed to 100% transcription.

The enriched visualization 500 may also include the provision for presenting emojis along with the textual data 508 for easy recognition of the emotion of the patient 302.

At step 422, the transcribed data is linked to a video-audio timeline by the Data Timeline 310. The Data Timeline 310 may also store the emotionally enhanced transcribed text along with the transcribed FIXtext 316 and the original information 308 provided by the patient 302. This will enable the therapist 106 to easily access the appropriate video-audio and its transcribed text. Using tools to “zoom-out” of transcribed text to identify “hot-spots”, the areas in which the text has been color-coded red, and then “zoom-in” to the text itself.

At step 424, the emotionally enhanced transcription system 300 may further use

Artificial Intelligence (AI) and Machine Learning (ML) 328 to search, track and analyze the correlation between the text (words) and the subtext (emotions) of the patient 302 in an audio or a video conversation. At step 426, the Artificial Intelligence (AI) and Machine Learning (ML) 328 also enables “on the fly” learning of the transcription system 300 by comparing the current the audio or the video conversation with previously stored conversations by the same person or different people. The process is completed at step 428.

FIG. 6 illustrates an exemplary system 600 for implementing various aspects of the invention. The system 600 includes a data processor 602, a system memory 604, and a system bus 616. The system bus 616 couples system components including, but not limited to, the system memory 604 to the data processor 602. The data processor 602 can be any of various available processors. The data processor 602 refers to any integrated circuit or other electronic device (or collection of devices) capable of performing an operation on at least one instruction, including, without limitation, Reduced Instruction Set Core (RISC) processors, CISC microprocessors, Microcontroller Units (MCUs), CISC-based Central Processing Units (CPUs), and Digital Signal Processors (DSPs). Furthermore, various functional aspects of the data processor 602 may be implemented solely as software or firmware associated with the processor. Dual microprocessors and other multiprocessor architectures also can be employed as the data processor 602.

The system bus 616 can be any of several types of bus structure(s) including the memory bus or memory controller, a peripheral bus or external bus, and/or a local bus using any variety of available bus architectures known to those of ordinary skill in the art.

The system memory 604 may include computer-readable storage media comprising volatile memory and nonvolatile memory. The non-volatile memory stores the basic input/output system (BIOS), containing the basic routines to transfer information between elements within the system 600. The nonvolatile memory can include, but not limited to, read only memory (ROM), programmable ROM (PROM), electrically programmable ROM (EPROM), electrically erasable programmable ROM (EEPROM), or flash memory. The volatile memory includes random access memory (RAM), which acts as external cache memory. RAM is available in many forms such as static RAM (SRAM), dynamic RAM (DRAM), synchronous DRAM (SDRAM), double data rate SDRAM (DDR SDRAM), enhanced SDRAM (ESDRAM), SynchLink™ DRAM (SLDRAM), Rambus® direct RAM (RDRAM), direct Rambus® dynamic RAM (DRDRAM), and Rambus® dynamic RAM (RDRAM).

The system memory 604 includes an operating system 606 which performs the functionality of managing the system 600 resources, establishing user interfaces, and executing and providing services for applications software. The system applications 608, modules 610 and data 612 provide various functionalities to the system 600.

The system 600 also includes a disk storage 614. Disk storage 614 includes, but is not limited to, devices like a magnetic disk drive, floppy disk drive, tape drive, Jaz drive, Zip drive, LS-100 drive, flash memory card, or memory stick. In addition, disk storage 614 can include storage media separately or in combination with other storage media including, but not limited to, an optical disk drive such as a compact disk ROM device (CD-ROM), CD recordable drive (CD-R Drive), CD rewritable drive (CD-RW Drive) or a digital versatile disk ROM drive (DVD-ROM).

A user enters commands or information into the system 600 through input device(s) 624. Input devices 624 include, but are not limited to, a pointing device (such as a mouse, trackball, stylus, or the like), a keyboard, a microphone, a joystick, a satellite dish, a scanner, a TV tuner card, a digital camera, a digital video camera, a web camera, and/or the like. The input devices 624 connect to the data processor 602 through the system bus 616 via interface port(s) 622. Interface port(s) 622 include, for example, a serial port, a parallel port, a game port, and a universal serial bus (USB).

The output devices 620 like monitors, speakers, and printers are used to provide output of the data processor 602 to the user. Another example, a USB port may be used as an input device 624 to provide input to the system 600 and to output information from system 600 to the output device 620. The output devices 620 connect to the data processor 602 through the system bus 616 via output adaptors 618. The output adapters 632 may include, for example, video and sound cards that provide a means of connection between the output device 620 and the system bus 616.

The system 600 can communicate with remote communication devices 628 for exchanging information. The remote communication device 628 can be a personal computer, a server, a router, a network PC, a workstation, a microprocessor-based appliance, a mobile phone, a laptop, a tablet, a paging device, a peer device or other common network node and the like.

Network interface 626 encompasses wire and/or wireless communication networks such as local-area networks (LAN) and wide-area networks (WAN). LAN technologies include Fiber Distributed Data Interface (FDDI), Copper Distributed Data Interface (CDDI), Ethernet, Token Ring and the like. WAN technologies include, but are not limited to, point-to-point links, circuit switching networks like Integrated Services Digital Networks (ISDN) and variations thereon, packet switching networks, and Digital Subscriber Lines (DSL).

The currently disclosed enhanced transcription system provides a TechTherapy—Technologically-Enhanced Therapy Mechanism. This mechanism may develop a platform specifically adapted for therapists of all kinds (psycho-therapy, life-coaches, spiritual leaders etc. . . . ).

The platform is based on BIOsubTEXT tools 318, datamining (DM), machine-learning (ML) and Artificial Intelligence (AI) 328 as well as Customer Relations Management (CRM) tools 334.

The BIOsubTEXT 318 may offer the therapist 106 glimpse of the emotional state of the patient 102 while the DM/ML/AI 328 may allow the therapist 106 the possibility to search, track and analyze the data acquired from the sessions.

Various other markets, apart from therapy, can benefit from the TechTherapy platform. These are usually markets in which communication is done through two or more people in frontal, telephone or video chats.

In all of these markets the technology of the platform can help make the communication more efficient.

The currently disclosed enhanced transcription system provides a TechTalk Platform. The TechTalk platform can be adapted to many markets, including:

The recruitments market: may use the TechTalk platform in interviews in order to make the screening process more efficient.

The pharmaceutical market: may use the TechTalk platform in meetings between pharma representatives and doctors.

The security markets: may use the TechTalk platform in investigations, high-security zones (airports, government buildings etc. . . . ).

The dating market: may use the TechTalk platform to develop an online dating platform which helps the users to connect (or not) in a much more efficient way.

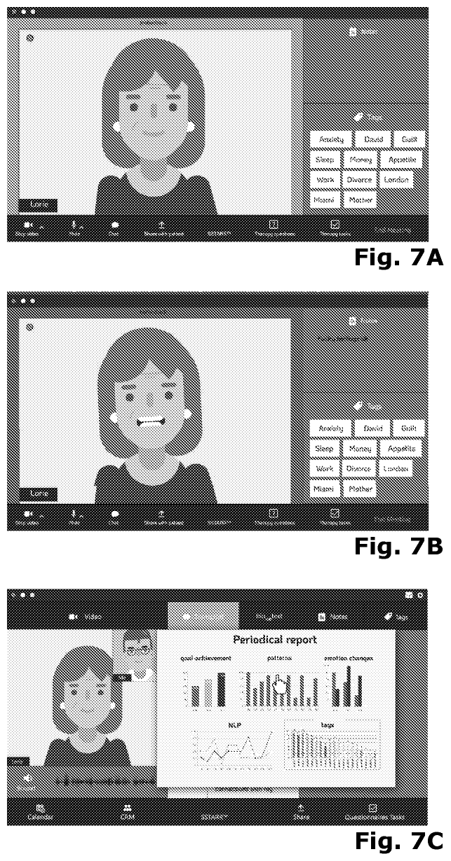

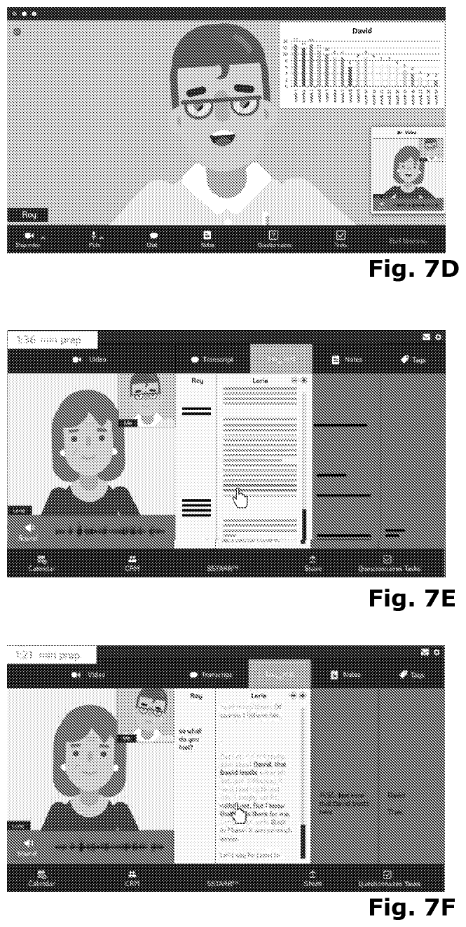

Referring now to FIGS. 7A-F, possible graphic user interfaces are presented for an emotion enhanced therapy visualization system.

In particular, FIG. 7A represents an in-session dashboard which may be used by a therapist either in real time or while reviewing a video of the session. Video of subjects may be framed by a color coded frame providing a biofeedback indicating their emotional state, for example via a voice sensitive analysis.

Whereas in FIG. 7A a green biofeedback frame indicates that the subject is relaxed, in FIG. 7B the frame has a red biofeedback frame indicating agitation on the part of the subject. The therapist may duly log the emotional state, select a suitable tag and take a manual note as required.

With reference to FIG. 7C, a therapist may be able to access from the dashboard analysis charts and graphs showing historical and statistical variation to provide an overview of the subject. Such charts may be shared with the subject or with other therapists as required to provide ongoing monitoring.

FIG. 7D indicates a possible subject side screen for use during a therapy session in which a therapist is able to share a selected chart to show illustrate the subject's progress.

FIGS. 7E and 7F indicates a review screen in which a therapist may navigate a color coded emotionally enhanced transcript of a therapy session and is easily able to zoom into areas of interest replaying the relevant video as required.

Technical and scientific terms used herein should have the same meaning as commonly understood by one of ordinary skill in the art to which the disclosure pertains. Nevertheless, it is expected that during the life of a patent maturing from this application many relevant systems and methods will be developed. Accordingly, the scope of the terms such as computing unit, network, display, memory, server and the like are intended to include all such new technologies a priori.

As used herein the term “about” refers to at least □10.%

The terms “comprises”, “comprising”, “includes”, “including”, “having” and their conjugates mean “including but not limited to” and indicate that the components listed are included, but not generally to the exclusion of other components. Such terms encompass the terms “consisting of” and “consisting essentially of.”

The phrase “consisting essentially of” means that the composition or method may include additional ingredients and/or steps, but only if the additional ingredients and/or steps do not materially alter the basic and novel characteristics of the claimed composition or method.

As used herein, the singular form “a”, “an” and “the” may include plural references unless the context clearly dictates otherwise. For example, the term “a compound” or “at least one compound” may include a plurality of compounds, including mixtures thereof.

The word “exemplary” is used herein to mean “serving as an example, instance or illustration”. Any embodiment described as “exemplary” is not necessarily to be construed as preferred or advantageous over other embodiments or to exclude the incorporation of features from other embodiments.

The word “optionally” is used herein to mean “is provided in some embodiments and not provided in other embodiments”. Any particular embodiment of the disclosure may include a plurality of “optional” features unless such features conflict.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween. It should be understood, therefore, that the description in range format is merely for convenience and brevity and should not be construed as an inflexible limitation on the scope of the disclosure. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6 as well as non-integral intermediate values. This applies regardless of the breadth of the range .

It is appreciated that certain features of the invention, which are, for clarity, described in the context of separate embodiments, may also be provided in combination in a single embodiment. Conversely, various features of the disclosure, which are, for brevity, described in the context of a single embodiment, may also be provided separately or in any suitable sub-combination or as suitable in any other described embodiment of the disclosure. Certain features described in the context of various embodiments are not to be considered essential features of those embodiments, unless the embodiment is inoperative without those elements.

Although the invention has been described in conjunction with specific embodiments thereof, it is evident that other alternatives, modifications, variations and equivalents will be apparent to those skilled in the art. Accordingly, it is intended to embrace all such alternatives, modifications, variations and equivalents that fall within the spirit of the invention and the broad scope of the appended claims .

Additionally, the various embodiments set forth hereinabove are described in term of exemplary block diagrams, flow charts and other illustrations. As will be apparent to those of ordinary skill in the art, the illustrated embodiments and their various alternatives may be implemented without confinement to the illustrated examples. For example, a block diagram and the accompanying description should not be construed as mandating a particular architecture, layout or configuration.

The presence of broadening words and phrases such as “one or more,” “at least,” “but not limited to” or other like phrases in some instances shall not be read to mean that the narrower case is intended or required in instances where such broadening phrases may be absent. The use of the term “module” does not imply that the components or functionality described or claimed as part of the module are all configured in a common package. Indeed, any or all of the various components of a module, whether control logic or other components, can be combined in a single package or separately maintained and can further be distributed in multiple groupings or packages or across multiple locations.

Furthermore, embodiments may be implemented by hardware, software, firmware, middleware, microcode, hardware description languages, or any combination thereof. When implemented in software, firmware, middleware or microcode, the program code or code segments to perform the necessary tasks may be stored in a computer-readable medium such as a storage medium. Processors may perform the necessary tasks.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present disclosure. To the extent that section headings are used, they should not be construed as necessarily limiting.

The scope of the disclosed subject matter is defined by the appended claims and includes both combinations and sub combinations of the various features described hereinabove as well as variations and modifications thereof, which would occur to persons skilled in the art upon reading the foregoing description.

1. A method 400 for generating emotionally enhanced transcription of non-textual data and an enriched visualization of the transcribed data, the method comprising:
capturing 408 the non-textual data of a speaker involved in a conversation;
transcribing 410 the non-textual data to generate a textual data;
obtaining 412 an emotional state of the speaker using one or more bio-feedback technologies;
combining 414 the generated transcribed textual data with the emotional state of the speaker to generate 416 the emotionally enhanced transcribed textual data; and
presenting 420 the emotionally enhanced transcribed textual data through an enriched visualization, wherein the enriched visualization includes color-coding, tempo-coding and weight-coding the emotionally enhanced transcribed textual data.

2. The method of claim 1, wherein the non-textual data comprises an audio, a video or a combination thereof.

3. The method of claim 1, wherein the non-textual data comprises an audio conversation 406 between the speaker and a user.

4. The method of claim 1, wherein the non-textual data comprises a video conversation 406 between the speaker and a user.

5. The method of claim 1, wherein the bio-feedback technologies include one or more of a Voice Sensitivity Analysis, Voice Stress Analysis, Facial Macro-Micro Expressions (FMME) technologies, Layered Voice Analysis, Infra-Red (heat) analysis and Oximeter (pulse) analysis.

6. The method of claim 1, wherein the Voice Sensitivity Analysis and the Voice Stress Analysis is used to analyse analyze the amount of stress in the voice of the speaker.

7. The method of claim 1, wherein the Facial Macro-Micro Expressions (FMME) technologies is used to identify different emotions exist bands which taps into subtext underlying spoken words of the non-textual data.

8. The method of claim 1, wherein color-coding the emotionally enhanced transcribed textual data comprises color-coding the transcribed textual data based on its level of uncertainty.

9. The method of claim 1, wherein color-coding the emotionally enhanced transcribed textual data comprises at least one enhancement selected from:
color-coding the transcribed textual data based on a level of stress of the speaker;
linking 422 the emotionally enhanced transcribed textual data to a video-audio timeline, wherein the video-audio timeline enables easy access of the non-textual data and its emotionally enhanced transcribed textual data; and
presenting the emotionally enhanced transcribed textual data through different color, size, weighting and spacing of the text.

10-11. (canceled)

12. The method of claim 1, wherein the enriched visualization further comprises zooming out of the transcribed textual data to identify hot-spot areas of mistakes and zooming in to the text in the hot-spot areas.

13. The method of claim 1 further comprises using Natural Language Processing (NLP) to fine tune the quality of the emotionally enhanced transcribed textual data.

14. The method of claim 1, further comprises using alternative therapy tools 418 to fine tune the quality of the emotionally enhanced transcribed textual data wherein the alternative therapy tools comprise one or more of a Natural Language Processing (NLP), Profile of Mood States (POMS), Hopkins Symptom Checklist (HSCL), Emotions Focused Therapy (EFT) and Positive and Negative Affect Schedule (PANAS).

15. The method of claim 1 further comprises using artificial intelligence 424 to search, track and analyze the correlation between the transcribed textual data and the emotions of the speaker in an audio or a video conversation.

16. The method of claim 15 further comprises using machine learning 426 to search, track and analyze the correlation between the transcribed textual data and the emotions of the speaker by comparing the audio or the video conversation with previously stored conversations.

17. The method of claim 1 further comprises using one or more emojis along with the transcribed textual data to identify the emotional state of the speaker.

18. The method of claim 1 further comprises a fL0Ow text mechanism, wherein the fL0Ow text mechanism is a Tempo-Spaced Text Mechanism configured to use the tempo of the sound-track and Micro-Expression analysis of the speaker in an audio or a video conversation to identify the emotional state of the speaker.

19. The method of claim 18, wherein the fL0Ow text mechanism includes presenting the emotionally enhanced transcribed textual data through different levels of font, letter and word spacing, boldness, italicizing, weighting of the text to identify the tempo of the speaker.

20. The method of claim 19, wherein the enriched visualization further comprises zooming out of the transcribed textual data to identify areas of different levels of tempo and zooming in to identify specific textual data related to the tempo.

21. The method of claim 1 further comprises providing a Customer Relations Management (CRM) tool 404, wherein the CRM tool enables multi-channel communication between the speaker and a user involved in an audio or a video conversation.

22. A system 300 for generating emotionally enhanced transcription of non-textual data and an enriched visualization of the transcribed data, the system comprising:
a receiving module 304 configured for receiving the non-textual data 308 of a speaker 302 involved in a conversation;
a transcription module 312 configured for transcribing the non-textual data to generate a textual data 316;
a bio-feedback module 314 configured for obtaining an emotional state 318 of the speaker using one or more bio-feedback technologies;
an analysis module 332 configured for combining the generated transcribed textual data with the emotional state of the speaker to generate the emotionally enhanced transcribed textual data; and
a visual presentation module 338 configured for presenting the emotionally enhanced transcribed textual data through an enriched visualization, wherein the enriched visualization includes color-coding, tempo-coding and weight-coding the emotionally enhanced transcribed textual data to identify mistakes in the transcribed data.

23-40. (canceled)
